SEQUENCE LISTING (6 sequences)

SEQ ID NO: 1 - DNA, 42 bp, Salmonella
  FIP inner primer for Salmonella invasion gene invA
gacgactggtactgatcgatagtttttcaacgtttcctgcgg

SEQ ID NO: 2 - DNA, 37 bp, Salmonella
  BIP inner primer for Salmonella invasion gene invA
ccggtgaaattatcgccacacaaaacccaccgccagg

SEQ ID NO: 3 - DNA, 22 bp, Salmonella
  F3 outer primer for Salmonella invasion gene invA
ggcgatattggtgtttatgggg

SEQ ID NO: 4 - DNA, 20 bp, Salmonella
  B3 outer primer for Salmonella invasion gene invA
aacgataaactggaccacgg

SEQ ID NO: 5 - DNA, 23 bp, Salmonella
  Loop F primer for Salmonella invasion gene invA
gacgaaagagcgtggtaattaac

SEQ ID NO: 6 - DNA, 23 bp, Salmonella
  Loop B primer for Salmonella invasion gene invA
gggcaattcgttattggcgatag

(19) United States
(12) Patent Application Publication
(10) Pub. No.: US 2022/0010369 A1
(43) Pub. Date: Jan. 13, 2022

(54) METHOD AND APPARATUS FOR PERFORMING A REAL-TIME COLORIMETRIC NUCLEIC ACID AMPLIFICATION ASSAY

(71) Applicant: FOUNDATION FOR RESEARCH AND TECHNOLOGY HELLAS, Heraklion, Crete (GR)

(72) Inventors: Georgios PAPADAKIS, Heraklion, Crete (GR); Electra GIZELI, Heraklion, Crete (GR); Alexandros PANTAZIS, Preveza (GR)

(73) Assignee: FOUNDATION FOR RESEARCH AND TECHNOLOGY HELLAS, Heraklion, Crete (GR)

(21) Appl. No.: 17/282,729

(22) PCT Filed: Oct. 31, 2019
(86) PCT No.: PCT/EP2019/079845

§ 371 Date: Apr. 2, 2021

(30) Foreign Application Priority Data
Oct. 31, 2018 (EP) 18203833.1

(51) Int. Cl. C12Q 1/6851; G01N 21/75

(52) U.S. Cl. CPC C12Q 1/6851; G01N 21/75

(57) ABSTRACT

Method and apparatus for performing a real-time colorimetric nucleic acid amplification assay wherein the heating of the liquid sample comprised in a reaction tube is carried out by bringing the bottom of the tube in thermal contact with a heating element. The real-time monitoring of the content of the reaction tube is carried out visually through the side wall of the tube, preferably by using a camera.

### FIELD OF THE INVENTION

The present invention relates to performing and monitoring in real-time colorimetric nucleic acid amplification assays.

### BACKGROUND OF THE INVENTION

Understanding of living organisms in terms of their molecular composition has led to the design of increasingly rapid and accurate diagnostic tests, mainly based on nucleic acid amplification and quantification. It has also led to a new trend in molecular diagnostics which is to have the actual diagnostic assay at the location where a sample is collected or a patient is treated (“point-of-need” testing). For point of-need testing, the design of increasingly rapid and accurate diagnostic assays, mainly based on nucleic acid amplification and quantification, is currently an emerging area with numerous applications in healthcare, agro/food safety, research etc. One of the most widespread assays is the polymerase chain reaction (PCR), which employs a polymerase enzyme which amplifies exponentially a specific target upon several numbers of heating cycles. Each cycle includes three steps; 1. Heating at 92-98° C. for double stranded DNA denaturation; 2. Specific primers annealing at 50-65° C.; and 3. Strand extension via the polymerase at 72° C. Efficient heating, a prerequisite for fast and correct products formation, is achieved by immersing of the reaction vessel (typically an Eppendorf tube) in a heating (metal) block, which is typically positioned on a heating element. The PCR, while the golden standard in lab-based nucleic acid detection and suitable for both end point and quantitative real-time detection, is not ideal for point-of-care or field based applications. This is because the PCR requires equipment for advanced temperature controlling and optical (fluorescent) monitoring that is either expensive and/or difficult to move around with the user. One alternative DNA amplification method, the loop mediated isothermal amplification (LAMP), is considered ideal for point-of-care (POC) applications since it requires only one temperature for amplification (65° C.) and can achieve visual detection of DNA through color change (colorimetric). In the LAMP-amplification colorimetric set ups, the reaction vessel is placed inside a heating block similar to the one used for PCR. Current formats of the LAMP colorimetric method are only based on end-point measurement. However, this poses two major drawbacks; firstly, color change can be often difficult to discern by naked eye, and secondly, end-point measurements can be used as qualitative tests (yes or no) which can be inadequate for many important applications. At the moment, there is no available solution to overcome these problems.

A key feature, which once solved would overcome the above obstacles and allow real-time colorimetric nucleic acid amplification is related to inventing a way/method to monitor the color change of the solution while the process takes place combined at the same time with efficient heating of the reaction. All currently used formats are based on the immersion of the reaction-vessel inside a heating block, which allows efficient amplification at the required elevated temperature. This means that visual monitoring, such as inspection and detection, can only be achieved through the top of the vessel. However, since during the amplification reaction the vessel is heated, part of the liquid sample is transformed into vapor which interferes with any possibility for real-time colorimetric monitoring from the top of the reaction vessel. For this reason, current formats of the LAMP colorimetric method are only based on end-point measurement, after allowing the sample to cool down to room temperature. This means that with the current formats, it is not possible to have real-time colorimetric monitoring.

### SUMMARY OF THE INVENTION

The present inventors have developed a method to convert the otherwise qualitative colorimetric nucleic acid amplification assays, for example LAMP assays, into quantitative and typically real time procedures. To achieve this, they have developed a method and an apparatus for performing a real-time colorimetric nucleic amplification assay. One important aspect of the present invention is the heating of a liquid sample without immersing the sample inside a heating block. This is achieved by bringing the bottom of the reaction tube containing the sample into thermal contact with a heating element. The method facilitates visual monitoring since it allows the visualisation of the content of the vessel through the side wall of the reaction tube. The present invention further provides a method for monitoring a colorimetric nucleic acid amplification assay by using the above heating method and by using visual monitoring, for example, with a camera.

In addition, the present invention provides an apparatus for performing a real-time colorimetric nucleic acid amplification assay.

### BRIEF DESCRIPTION OF THE DRAWINGS

FIG. 1 shows the different parts of a reaction tube and the footprint of the bottom of a reaction tube.

FIG. 2 shows a schematic representation of an apparatus according to the present invention.

FIG. 3 shows an embodiment of an apparatus according to the present invention

FIG. 4 shows the positioning of the reaction tubes in an apparatus according to the present invention.

FIG. 5 shows experimental data of a LAMP assay performed according to the present invention and using phenol red and hydroxyl naphthol blue (HNB) as an indicator coloured substance.

FIG. 6 shows experimental data of a LAMP assay performed according to the present invention, using phenol red as an indicator coloured substance under different amounts of pressure.

### DETAILED DESCRIPTION OF THE INVENTION

In nucleic acid amplification assays, the liquid sample is typically contained in a reaction tube, often called Eppendorf tube. Typically, such a tube is made of a polymer material, such as polypropelene and has a volume from 10 μl to 200 μl. Reaction tubes similar to the commercially available Eppendorf tubes can be manufactured using other optically transparent/translucent materials and 3D-printing.

In the systems of the prior art, in order to heat the liquid phase to the required temperature, the tube is immersed inside a heating block.

The present inventors have now surprisingly found that immersion of the vessel inside a heating block is not necessary for efficient amplification and that the liquid phase can be heated by bringing the bottom of the tube containing the liquid phase in thermal contact with a heating element.

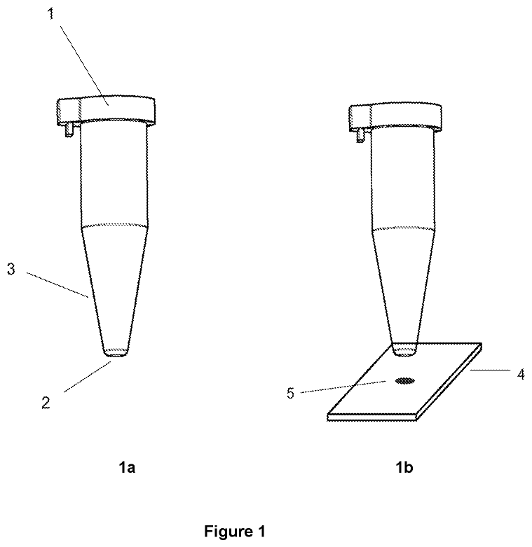

According to the present invention the “top of the tube” or the “top of the reaction tube” is the opening through which the liquid sample is loaded into the tube. The “bottom of the tube” or the “bottom of the reaction tube” is the part of the tube which is opposite to the top of the tube. FIG. 1a shows the top (1), bottom (2) and side wall (3) of a reaction tube.

According to the present invention, the reaction tube can be positioned directly on the surface of a heating element. Moreover, the heating element may comprise a surface, made of a thermally conductive material, on which the reaction tube can be positioned. The heating element is typically a resistive heater or a peltier element.

Preferably, when placed on the heating element, the longitudinal axis of the tube forms an angle with the heating element which is from 60 to 120 degrees. More preferably, the angle is substantially a right angle.

For an effective nucleic acid amplification assay, effective heating of the liquid phase is one of the most important prerequisites. The prior art teaches that for effective heating of the liquid phase, a large area of reaction tube must be in thermal contact with a heated body. For this reason, the reaction tube must be immersed in a metal block which surrounds almost the entire tube and is in thermal contact with the bottom and the side wall of the tube. This means that the prior art teaches that for effective heating, almost the whole surface of the tube must be in thermal contact with a heated body. It has now unexpectedly been found that effective heating can be achieved by bringing only the bottom of the tube, i.e., a very small part of the tube, in thermal contact with a heating element.

Preferably, the area of the tube which is in thermal contact with the heating element is up to 12 mm². More preferably, the area of the tube which is in thermal contact with the heating element is up to 6 mm². Even more preferably, the area of the tube which is in thermal contact with the heating element is up to 3 mm². The area of the tube which is in thermal contact with the heating element can be determined by establishing the footprint of the bottom of the tube, as shown in FIG. 1b. First, the bottom of the tube is coloured, for example with a marker and then the tube is pressed on a piece of paper (4) to obtain the circular footprint (5) of the bottom of the tube. The area of the footprint is the area of the tube which is in thermal contact with the heating element.

The present inventors have also surprisingly found that the effectiveness and efficiency of the heating method of the present invention can be increased by applying pressure on the tube towards the heating element. For example, by doing this, the amount of time needed before the amplification reaction begins is significantly shortened. This aspect is very important, especially in point-of-care applications, where an assay has to be carried out in the shortest possible time. Preferably, the pressure applied on the reaction tube is such that the pressure applied on the heating element by the tube is from 0.4 MPa to 15 MPa. More preferably, the pressure applied on the heating element by the tube is from 1 MPa to 10 MPa. Even more preferably, the pressure applied on the heating element by the tube is from 1 MPa to 3 MPa. The pressure may be adjusted by different means, well known to a person skilled in the art. For example, the pressure may be adjusted by applying different weights on the tube, or by using a system of screws, or by using a system of magnets. The pressure can be determined by methods well known to a person skilled in the art. For example, the pressure can be calculated by measuring the perpendicular force applied on the heating element by the tube, then measuring the area of the tube which is in contact with the heating element, as described above, and finally dividing the value of the perpendicular force by the area.

The nucleic acid amplification assay may be, for example, an isothermal amplification assay such as transcription mediated amplification, nucleic acid sequence-based amplification, signal mediated amplification of RNA technology, strand displacement amplification, rolling circle amplification, LAMP, isothermal multiple displacement amplification, recombinase polymerase amplification, helicase-dependent amplification, single primer isothermal amplification, and circular helicase-dependent amplification. Preferably, the assay is a LAMP assay.

The effectiveness of the heating method of the present invention is the same as that of the prior art methods, in which the tube is immersed in the heating block. On the other hand, the heating method of the present invention provides greater efficiency towards energy-consumption, because the amount of energy required to heat the liquid phase is less.

Another advantage of the heating method of the present invention is that, when the tube is made of a translucent or a transparent material, the content of the tube is visible not only from the top of the tube but more importantly through the side wall. This means that the visual monitoring of a parameter of the assay, such as the colour change in a colorimetric LAMP assay, becomes possible during the run of the assay. This is because the evaporation of the sample during the amplification reaction does not interfere with the monitoring. Therefore, the heating method of the present invention enables real-time monitoring of the assay.

Therefore, another aspect of the present invention is a method for performing a real-time colorimetric nucleic acid amplification assay, wherein the method comprises heating the liquid phase by bringing the bottom of the tube containing the liquid phase in thermal contact with a heating element and utilizing real-time visual monitoring of a parameter of the assay.

The visual monitoring includes the monitoring by the eye of the user as well as the monitoring by a camera. Preferably, the monitoring is carried out by a camera.

According to a preferred embodiment, the present invention provides a method in which a digital colour camera is used to monitor in real-time changes in the colour of the liquid sample due to the formation of, reaction of, or change in colour of a coloured substance.

According to this preferred embodiment, the method comprises using a digital colour camera to record one or more images of the liquid sample, processing for each image one or more of red channel data, green channel data and blue channel data obtained from the image and thereby obtaining a parameter of the assay.

In an endpoint assay, the method typically involves recording and processing of a single image. When the method involves recording and processing data obtained from a plurality of images, the series of images may form a video.

A parameter of the assay could be for example the presence of an analyte or its amount or the efficiency of a nucleic acid amplification reaction, such as LAMP.

The couloured substance comprises a substance which is formed, or consumed, or changes its colour during the assay. The change in colour may for example occur in response to a change in pH or in response to a chemical reaction. The colour change may involve the change from one colour to another, or from a colour to transparent, or vice versa.

Examples of coloured substances used in nucleic acid amplification assays include hydroxynaphtol blue (HNB), phenol red, calcein, crystal violet, SYBR green I, cresol red, neutral red, m-cresol purple, gold nanoparticles, polydiacetylene (PDA) liposomes and other substances well known to a person skilled in the art.

The recording of the images and/or the processing of the data obtained from the image may comprise one or more image processing steps, such as one or more of colour mode conversion, image calibration and gamma correction.

Typically, the image comprises pixels and the processing step comprises extracting the level of light, for example its intensity, of one or more of red, green and blue from pixels of the image and thereby obtaining the one or more of red, green and blue channel data respectively. The level (e.g. intensity) of light recorded could be represented in a pixel of the image. The level could be an intensity, for example the intensity recorded by the channels of the camera. The level may be an RGB colour value, e.g. 8 bit, 16 bit, 24 bit or 32 bit.

Typically, a pixel of an image recorded by the digital camera comprises elements each representing the different colour components of the image. The colour components are typically red, green and blue, corresponding to the red, green and blue channels respectively, although the camera may record and/or the images may be stored using data according to a different colour model, such as CIECAM02 which uses lightness, chroma and hue as dimensions.

The images may be multi-pixel images of regions of the liquid phase. The images may be single-pixel images of regions of the liquid phase, which may for example be obtained using an optical fibre, or an optical fibre per colour channel. The images may be displayed on a computer screen for example, or in any other manner known to the person skilled in the art of displaying images.

The processing step of the present invention may comprise calculating a value from the one or more of red channel data, green channel data and blue channel data, for example by carrying out a mathematical operation.

Preferably, the processing step comprises calculating the difference between two out of three of red channel data, green channel data and blue channel data. More preferably, the processing step comprises calculating the difference between the red channel data and the green channel data, or between the blue channel data and the green channel data.

It is possible that a video image recording of the liquid phase is made using the digital camera. Such a video image recording can then be broken down into its constituent images, using for example a hardware processor. A video image may enable monitoring a liquid phase assay in real-time.

Another aspect of the present invention is an apparatus for performing the method of the present invention. Thus, the present invention provides an apparatus for performing a real-time colorimetric nucleic acid amplification assay, wherein the apparatus comprises

a heating element,
a reaction tube made of a translucent or a transparent material and arranged such that the bottom of the tube is in thermal contact with the heating element,
a digital colour camera arranged such that it can record images through the side wall of the reaction tube and
a processing unit configured to process an image obtained by the digital colour camera and to process one or more of red, green and blue channel data of the image to thereby obtain a parameter of the assay.

Preferably, the apparatus further comprises means for applying pressure on the reaction tube towards the heating element.

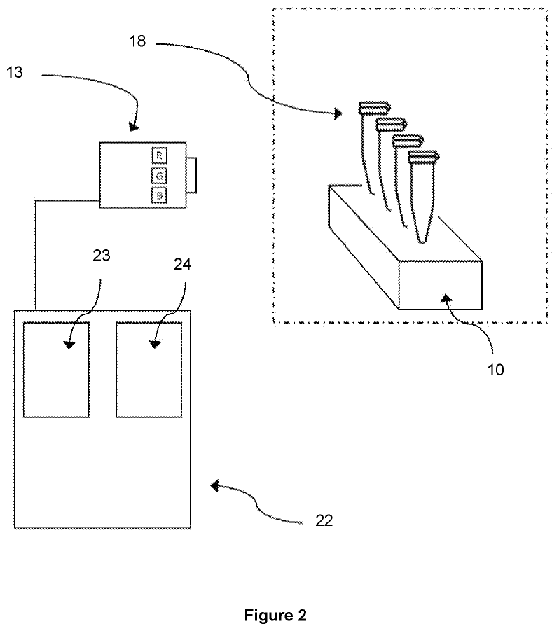

A schematic representation of an apparatus according to the present invention is shown in FIG. 2. The nucleic acid amplification is carried out in reaction tubes (18), which are positioned on a heating element (10) so that only the bottom of the tubes is in thermal contact with the heating element. The camera (13) is arranged such that it can record images through the side wall of the reaction tubes. The output of the camera (13) is passed onto a processing unit (22), such as a computer, having a processor (23) and a memory (24) for storing image data and a computer program executed by the processor. The processing unit (22) can be a separate unit, as shown in FIG. 2 or can be an integral component of a camera or other device including a camera.

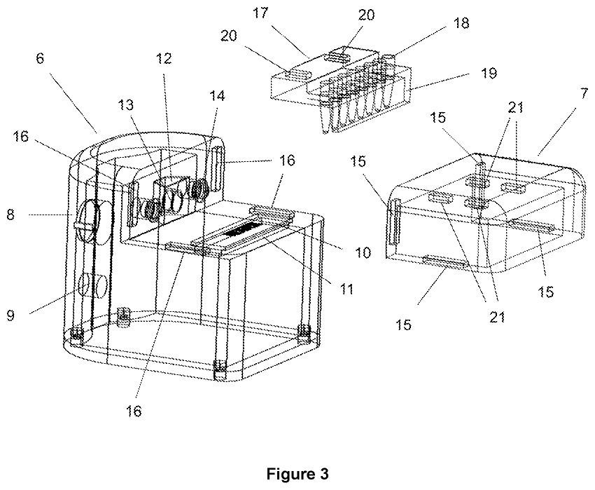

FIG. 3 shows an embodiment of an apparatus according to the present invention which can be used for performing and monitoring a nucleic acid amplification assay, such as a colorimetric LAMP assay.

The apparatus comprises a main housing unit (6) which comprises a main switch (8) and a power supply socket (9). It further comprises a heating element (10), which is attached to the main housing unit (6) through a heating element holder (11). The main housing unit (6) further comprises a camera holder (12) for receiving a camera module (13), and LED lights (14) for illuminating the content of the reaction tube. The main housing (6) further comprises a microprocessor and an electronic board (not shown) for processing the images recorded by the camera.

The apparatus further comprises a cover (7), which comprises four large magnets (15) which engage with corresponding large magnets (16) of the main housing unit (6) and secure the cover (7) it its intended position, when placed on the main housing unit (6).

The apparatus further comprises a reaction tube holder (17) which comprises slots for receiving the reaction tubes (18). The reaction tubes (18) are made of a translucent material. The reaction tube holder (17) further comprises a background wall (19), having a white colour on its side facing the tubes (18), which facilitates monitoring of the colour change during the assay.

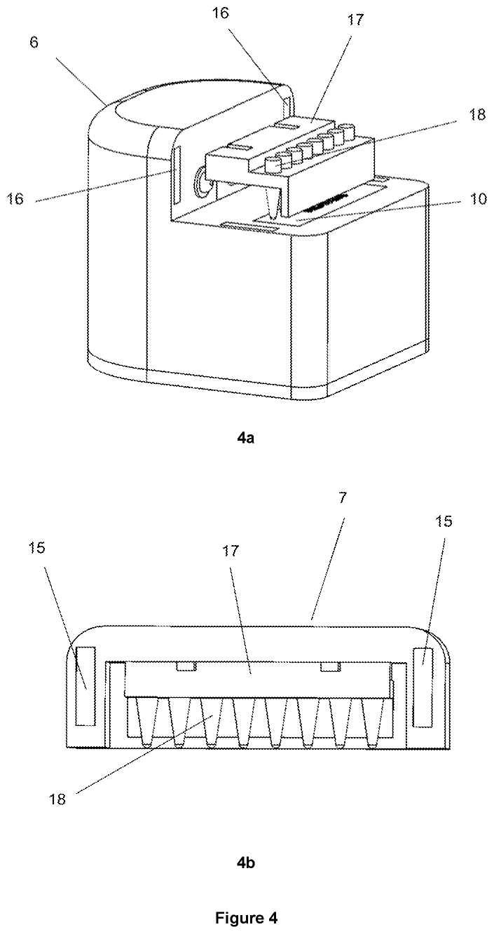

For the performance of the assay, the liquid sample is added to the reaction tubes (18) which are placed in the corresponding slots. The reaction tube holder (17) is placed on the heating element (10) (FIG. 4a) so that the bottom of the reaction tubes (18) comes into thermal contact with the heating element (10). This allows the camera to view the liquid phase through the side wall of the tubes (FIG. 4b). The cover (7) is secured in its position by engaging the large magnets (15) of the cover with the large magnets (16) of the main housing unit (6). Thereby the reaction tube holder (17) is secured in its position by small magnets (20) which engage with corresponding small magnets (21) of the cover (7). The pressure exerted by the bottom of the tubes (18) on the heating element (10) can be adjusted by modifying the size and/or number of the large magnets (15) and (16) on the cover (7) and the main housing unit (6).

The digital camera records images during the assay, which are passed on to the processing unit. The processing unit is configured to calculate the difference between red and green or blue and green channels. Changes in these values with time are processed to calculate the change in colour of the liquid phase.

### EXAMPLES

### Example 1

Bacterial cells resuspended in PBS buffer were lysed for 1 min at 95° C. The Salmonella invasion gene invA was targeted by a set of six primers, two outer (F3 and B3), two inner (FIP and BIP) and two loop (Loop-F and Loop-B).

FIP: GACGACTGGTACTGATCGATAGTTTTTCAACGTTTCCTGCGG
BIP: CCGGTGAAATTATCGCCACACAAAACCCACCGCCAGG
F3: GGCGATATTGGTGTTTATGGGG
B3: AACGATAAACTGGACCACGG
Loop F: GACGAAAGAGCGTGGTAATTAAC
Loop B: GGGCAATTCGTTATTGGCGATAG

The LAMP reagent mix in a total volume of 25 μl contained 12.5 μl of the standard or the colorimetric WarmStart 2×Master Mix (New England BioLabs), which uses phenol red as coloured substance, 1.8 μM FIP and BIP, 0.1 μM F3 and B3, 0.4 μM Loop-F and Loop-B, and 1 μl lysed cells in PBS. The reactions were carried out in reaction tubes placed in an apparatus as that shown in FIGS. 3-4. Monitoring of the assay was carried out by recording images of the content of the tubes by the digital camera of the apparatus.

### Example 2

The resulting images from Example 1 are processed as follows.

Firstly, a sequence of images of the liquid phase are obtained, as described above. Original red (R), green (G) and blue (B) channels from the camera may be subjected to image procession, including for example gamma correction, colour adjustment and so forth. Secondly, the red, green and blue channel data is extracted from one or more pixels of each image.

In a third step, for each of the red, green and blue channels, for each time point, the initial value of the red, green and blue channel is subtracted, to give data starting from zero. The value which is subtracted may be the value of red, green or blue respectively at the first time point, although typically the values from a number of initial time points may be averaged, or a curve may be plotted and the time zero axis intercept calculated and subtracted.

Next, the difference between two of the channels is calculated. In the case of the example protocol below with phenol red the difference between the green and the blue values is calculated (i.e. the blue values obtained from the previous step are subtracted from the green values). This takes place for each time point. With HNB, the difference between the green and the red values is calculated.

Next, the differences are optionally plotted, and then analysed, to determine one or more parameters of the assay. The time at which the calculated difference value increases to above a threshold can be used to determine the presence, or amount of analyte, for example with reference to a control (which may be a parallel measurement of one or more reactions with known concentrations of analyte and/or pre-stored data). The variation in the difference values with time can also be analysed to establish the efficiency of the amplification reaction and also to improve an estimate of the amount of analyte, for example, by extrapolation back to the start time.

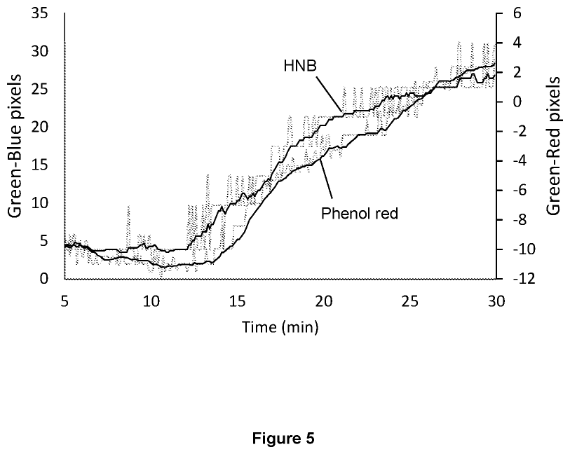

FIG. 5 shows results from a comparison of real-time monitoring of LAMP amplification of 2 positive (infected/10 bacteria present) samples using 2 different coloured substances; phenol red, which is a pH indicator and HNB which is a metal binding indicator. Images of the liquid phase have been recorded and analyzed automatically as a function of time as the assays progress. The difference between green and blue channels or green and red channels is calculated in each case. The pressure applied by the reaction tubes on the heating element during the assay was 2 MPa.

FIG. 5 shows the real-time curves during LAMP amplification of 10 bacteria using phenol red or HNB color indicators. The difference between the green and blue channels has been calculated for the phenol red indicator while the difference between the green and the red pixels is used for HNB. The plots show that in both cases a positive signal can be observed before the 15^{th} minute of the reaction. It is therefore possible to determine whether an analyte (in this case Salmonella cells) is or is not present and the size of the difference at a given time can be used to determine the amount of the analyte, for example in comparison to one or more controls.

### Example 3

This example illustrates a second set of results from a comparison of a positive (infected/Salmonella present) sample against a negative (non-infected/Salmonella not present) sample by using the image processing method of Example 2. The coloured substance which has been used in this case is phenol red. Images of the liquid phase have been recorded as a function of time as the assay progresses. The difference between green and blue channels is calculated.

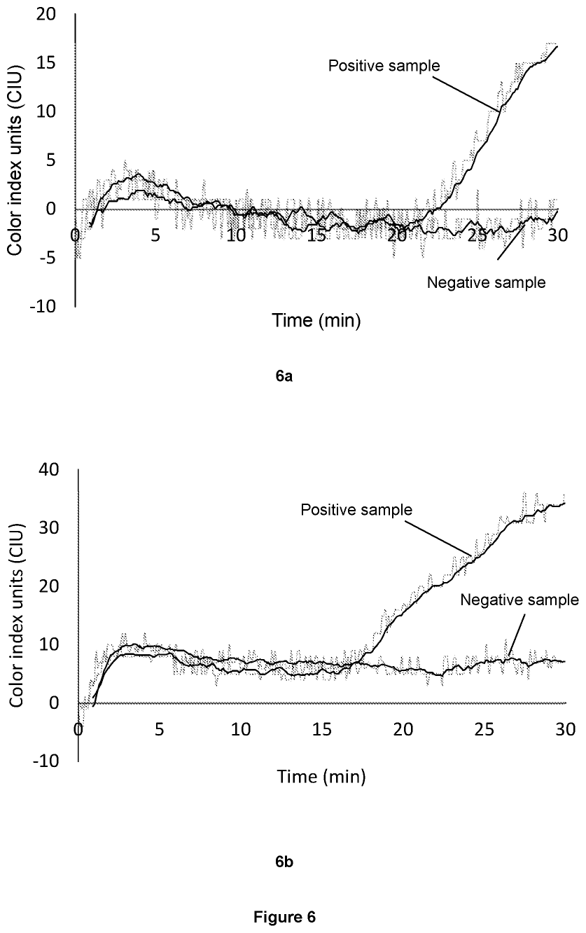

FIG. 6a shows real time colorimetric LAMP detection of Salmonella cells (positive vs negative sample) carried out in reaction tubes placed in an apparatus as that shown in FIGS. 3-4. The pressure applied by the tubes on the heating element was 0.4 MPa. The change in the color of the positive curve begins at the 23rd minute. The maximum color change (color index units) measured at minute 30 is 17 units.

FIG. 6b shows an example of real time colorimetric LAMP detection of Salmonella cells (positive vs negative sample) carried out in the same manner as explained in the previous paragraph. However, in this case, the pressure applied by the tubes on the heating element was 2 MPa. The change in the color of the positive curve begins at the 17th minute. The maximum color change (color index units) measured at minute 30 is 28 units. A 6 min earlier detection in the case of an exponential amplification assay may result in an improved sensitivity of 1-2 orders of magnitude.

1. A method for performing a real-time colorimetric nucleic acid amplification assay in a liquid sample comprised in a reaction tube made of a translucent or a transparent material, wherein the method comprises
heating the liquid sample by bringing the bottom of the reaction tube in thermal contact with a heating element and
visually monitoring a parameter of the assay through the side wall of the reaction tube.

2. The method according to claim 1, wherein the method further comprises applying pressure on the reaction tube towards the heating element.

3. The method according to claim 2, wherein the pressure applied by the reaction tube on the heating element is from 0.4 MPa to 15 MPa.

4. The method according to claim 3, wherein the pressure applied by the reaction tube on the heating element is from 1 MPa to 10 MPa.

5. The method according to claim 4, wherein the pressure applied by the reaction tube on the heating element is from 1 MPa to 3 MPa.

6. The method according to claim 1, wherein the longitudinal axis of the reaction tube forms an angle of from 60 to 120 degrees with the heating element.

7. The method according to claim 6, wherein the longitudinal axis of the reaction tube forms an angle of 90 degrees with the heating element.

8. The method according to any claim 1, wherein the area of the reaction tube which is in thermal contact with the heating element is up to 12 mm².

9. The method according to claim 8, wherein the area of the reaction tube which is in thermal contact with the heating element is up to 6 mm².

10. The method according to claim 9, wherein the area of the reaction tube which is in thermal contact with the heating element is up to 3 mm².

11. The method according to claim 1, wherein the reaction tube is made of a transparent material.

12. The method according to claim 1, wherein the monitoring is carried out by a digital camera.

13. The method according to claim 1, wherein the nucleic acid amplification assay is an isothermal nucleic acid amplification assay.

14. The method according to claim 13, wherein the nucleic acid amplification assay is a loop mediated isothermal amplification assay.

15. An apparatus for performing a real-time colorimetric nucleic acid amplification assay according to the method of claim 1, wherein the apparatus comprises
a heating element,
a reaction tube made of a translucent or a transparent material and arranged such that the bottom of the tube is in thermal contact with the heating element,
a digital colour camera arranged such that it can record images through the side wall of the reaction tube and
a processing unit configured to process an image obtained by the digital colour camera and to process one or more of red, green and blue channel data of the image to thereby obtain a parameter of the assay.

16. The apparatus according to claim 15, wherein the apparatus further comprises means for applying pressure on the reaction tube towards the heating element.

17. The apparatus according to claim 15, wherein the pressure applied by the reaction tube on the heating element is from 0.4 MPa to 15 MPa.

18. The apparatus according to claim 17, wherein the pressure applied by the reaction tube on the heating element is from 1 MPa to 10 MPa.

19. The apparatus according to claim 18, wherein the pressure applied by the reaction tube on the heating element is from 1 MPa to 3 MPa.

20. The apparatus according to claim 15, wherein the reaction tube is made of a transparent material.

21. The apparatus according to claim 15, wherein the area of the reaction tube which is in thermal contact with the heating element is up to 12 mm².

22. The apparatus according to claim 21, wherein the area of the reaction tube which is in thermal contact with the heating element is up to 6 mm².

23. The apparatus according to claim 22, wherein the area of the reaction tube which is in thermal contact with the heating element is up to 3 mm².

24. The apparatus according to claim 15, wherein the longitudinal axis of the reaction tube forms an angle of from 60 to 120 degrees with the heating element.
